SEQUENCE LISTING (2 sequences)

SEQ ID NO: 1 - RNA, 22 nt, synthetic construct
  source 1..22: mol_type = other RNA
  misc_feature 1..22: note = guide strand miR-15a mimic
  modified_base 1: mod_base = OTHER; note = uracil or 5-fluorouracil
  modified_base 11: mod_base = OTHER; note = uraci or 5-fluorouracil
  modified_base 14: mod_base = OTHER; note = uracil or 5-fluorouracil
  modified_base 17..19: mod_base = OTHER; note = uracil or 5-fluorouracil
  modified_base 21: mod_base = OTHER; note = uracil or 5-fluorouracil
  modified_base 2: mod_base = OTHER; note = adenine or 6-mercaptopurine
  modified_base 5: mod_base = OTHER; note = adenine or 6-mercaptopurine
  modified_base 8: mod_base = OTHER; note = adenine or 6-mercaptopurine
  modified_base 10: mod_base = OTHER; note = adenine or 6-mercaptopurine
  modified_base 12..13: mod_base = OTHER; note = adenine or 6-mercaptopurine
tagcagcacataatggtttgtg

SEQ ID NO: 2 - RNA, 22 nt, synthetic construct
  source 1..22: mol_type = other RNA
  misc_feature 1..22: note = passenger strand miR-15a mimic
  modified_base 2: mod_base = OTHER; note = adenine or 6-mercaptopurine
  modified_base 7: mod_base = OTHER; note = adenine or 6-mercaptopurine
  modified_base 9: mod_base = OTHER; note = adenine or 6-mercaptopurine
  modified_base 22: mod_base = OTHER; note = adenine or 6-mercaptopurine
caggccatattgtgctgcctca

(19) United States
(12) Patent Application Publication
(10) Pub. No.: US 2024/0150765 A1
(43) Pub. Date: May 9, 2024

(54) MERCAPTOPURINE-MODIFIED MIRNA FOR TREATING CANCER

(71) Applicant: Curamir Therapeutics, Inc., Woburn, MA (US)

(72) Inventor: Andrew Fesler, Woburn, MA (US)

(21) Appl. No.: 18/504,849

(22) Filed: Nov. 8, 2023

Related U.S. Application Data
(60) Provisional application No. 63/423,651, filed on Nov. 8, 2022.

(51) Int. Cl. C12N 15/113; A61P 35/00

(52) U.S. Cl. CPC C12N 15/1135; A61P 35/00; C12N 2310/141; C12N 2310/335

(57) ABSTRACT

A composition that includes a micro-RNA mimic containing one or more adenine bases in which at least one adenine base is replaced by 6-mercaptopurine. Also provided is a method for killing a cancer cell by contacting it with a composition that includes a micro-RNA mimic containing 6-mercaptopurine and 5-fluorouracil in which the micro-RNA mimic is miR-15a, miR-16, miR-129, miR-194, miR-192, miR-139, miR-140, or miR-145. Further, disclosed is a method for treating cancer by administering a composition containing a micro-RNA mimic having a guide strand and a passenger strand in which at least one adenine base in the guide strand or the passenger strand has been replaced by 6-mercaptopurine.

### CROSS-REFERENCE TO RELATED APPLICATIONS

This application claims the benefit of U.S. Provisional Patent Application Ser. No. 63/423,651, filed on Nov. 8, 2022. The entire contents of the above application is incorporated herein by reference.

### SEQUENCE LISTING XML

A computer readable file containing a sequence listing is being electronically co-filed herewith via EFS-Web. The computer readable file, submitted under 37 CFR § 1.831(e), will also serve as the copy required by 37 § CFR 1.831(c). The file (filename “CUTH-003-101 SEQ LIST.XML”) was created on Nov. 3, 2023 and has a size of 11,341 bytes.

The content of the computer readable file is hereby incorporated by reference in its entirety.

### BACKGROUND

Acute myeloid leukemia (“AML”) presents a significant clinical challenge. AML cases, incidence, and deaths have been rising worldwide over the past 30 years and clinical outcomes remain poor. AML is most prevalent in elderly populations, with greater than 75% of cases occurring in patients over 65 years old. The 5-year overall survival rate for these patients is approximately 24% with median survival of 8.5 months. Traditional chemotherapeutic approaches based on anthracycline and cytarabine are somewhat effective for treating AML. However, elderly patients are not candidates for intensive chemotherapy.

Mercaptopurine (“6-MP”) is an antimetabolic agent that inhibits DNA and RNA synthesis, resulting in the killing of rapidly proliferating cells. 6-MP has been used as a chemotherapy agent to treat cancer, specifically acute lymphocytic leukemia (“ALL”), acute promyelocytic leukemia (“APL”), and acute myeloid leukemia (“AML”), as well as Crohn's disease and ulcerative colitis.

Aside from chemotherapy, cancer treatments based on micro-RNA (“miRNA”) are being developed. miRNAs are short non-coding RNAs with important roles in regulating gene expression. Individual miRNAs can inhibit the expression of many different target genes through imperfect base pairing to their 3′ untranslated region.

The importance of miRNAs in cancer was first identified in chronic lymphocytic leukemia, in which miRNA 15a (“miR-15a”) and miRNA 16 (“miR-16”) were found to have reduced expression. Since that discovery, miRNAs have been shown to play a significant role in many different cancer types.

There remains a need to develop new therapeutic approaches having reduced toxicity with the goal of extending survival for patients suffering from AML and other leukemias.

### SUMMARY

To meet the above need, a composition is provided that includes a miRNA mimic containing one or more adenine bases in which at least one adenine base is replaced by 6-mercaptopurine (“6-MP”).

Also disclosed is a method for killing a cancer cell by contacting it with a composition that includes a miRNA mimic containing 6-MP and 5-fluorouracil (“5-FU”) in which the miRNA mimic is miR-15a, miR-16, miR-129, miR-194, miR-192, miR-139, miR-140, or miR-145.

Further, provided is a method for treating cancer by administering to a subject an effective amount of a composition containing a miRNA mimic having a guide strand and a passenger strand that each contains one or more adenine bases in which at least one adenine base in the guide strand or at least one adenine base in the passenger strand is replaced by 6-MP.

The details of one or more embodiments of the invention are set forth in the description below. Other features, objects, and advantages of the invention will be apparent from the description, from the drawings, and from the claims.

### BRIEF DESCRIPTION OF THE DRAWINGS

The description below refers to the accompanying drawings, of which:

FIG. 1A depicts a modified miRNA mimic in which adenine bases in the guide strand (underlined) have been replaced with 6-MP. G=guide strand (SEQ ID NO: 1); P=passenger strand (SEQ ID NO: 2).

FIG. 1B depicts a modified miRNA mimic in which adenine bases in the passenger strand (underlined) have been replaced with 6-MP. G=guide strand (SEQ ID NO: 1); P=passenger strand (SEQ ID NO: 2)

FIG. 1C. depicts a modified miRNA mimic in which adenine bases in the passenger strand and the guide strand (underlined) have been replaced with 6-MP. G=guide strand (SEQ ID NO: 1); P=passenger strand (SEQ ID NO: 2).

FIG. 1D depicts a modified miRNA mimic in which adenine bases in the passenger strand (underlined) have been replaced with 6-MP and uracil bases in the guide strand (double underlined) have been replaced with 5-FU. G=guide strand (SEQ ID NO: 1); P=passenger strand (SEQ ID NO: 2).

FIG. 1E depicts a modified miRNA mimic in which adenine bases in the passenger strand and the guide strand (underlined) have been replaced with 6-MP and uracil bases in the guide strand (double underlined) have been replaced with 5-FU. G=guide strand (SEQ ID NO: 1); P=passenger strand (SEQ ID NO: 2).

FIG. 1F depicts a modified miRNA mimic in which adenine bases in the guide strand (underlined) have been replaced with 6-MP and uracil bases in the guide strand (double underlined) have been replaced with 5-FU. G=guide strand (SEQ ID NO: 1); P=passenger strand (SEQ ID NO: 2).

### DETAILED DESCRIPTION

As set out above in the SUMMARY section, a composition is disclosed that includes a miRNA mimic containing one or more adenine bases in which at least one adenine base is replaced by 6-MP. In the composition, the miRNA mimic can include a guide strand and a passenger strand. The 6-MP can be present in the guide strand, in the passenger strand, or in both strands.

The composition can include a pharmaceutically acceptable excipient or carrier. The excipient can be a cationic polymer such as polyethyleneimine, and the carrier can be a lipid carrier, e.g., or a lipid nanoparticle. Additional examples of excipients and carriers are set forth in Ju et al., US Patent Application Publication 2019/0062754 (“Ju”), the contents of which are hereby incorporated by reference in its entirety.

In certain compositions, a single adenine base in the guide strand, in the passenger strand, or in both strands is replaced by 6-MP. In a particular composition, all of the adenine bases in the passenger strand are replaced with 6-MP. In another composition, all of the adenine bases in the guide strand are replaced with 6-MP. Also within the scope of the invention is a composition in which all adenine bases in both the passenger strand and the guide strand are replaced by 6-MP

The compositions described above can include a guide strand that contains one or more uracil bases in which at least one uracil base is replaced by 5-halouracil. The 5-halouracil can be 5-bromouracil, 5-chlorouracil, 5-iodouracil, or 5-fluorouracil. In a particular composition the 5-halouracil is 5-fluorouracil (“5-FU”).

In certain compositions, a single uracil base in the guide strand is replaced with 5-FU. In other compositions, all of the uracil bases in the guide strand are replaced with 5-fluorouracil.

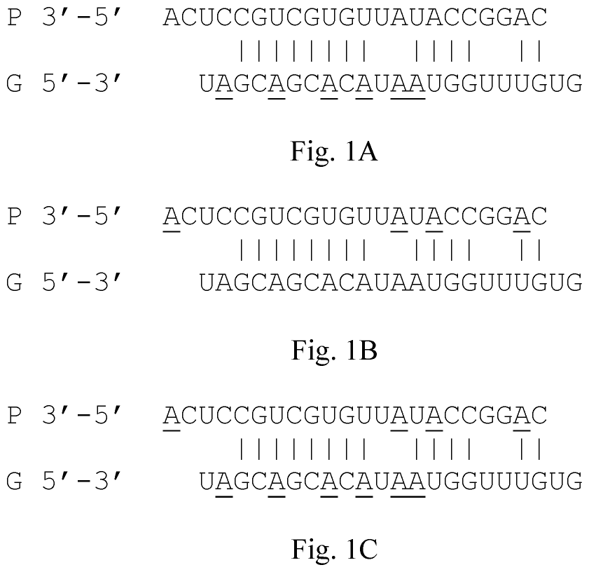

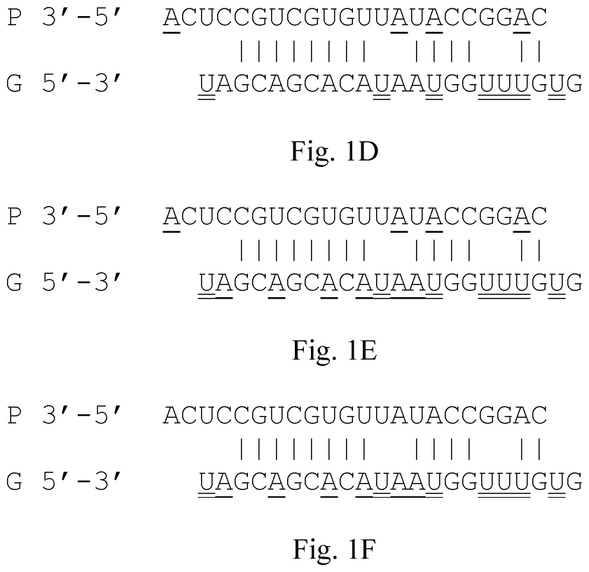

Any combination of 6-MP- and 5-FU-modified guide strands and passenger strands can be included in the composition. See FIGS. 1A-1F. For example, one composition contains a guide strand and a passenger strand both having all adenine bases replaced by 6-MP, together with all uracil bases in the guide strand replaced by 5-FU. In another composition, all of the adenine bases in the passenger strand are replaced by 6-MP, none of the adenine bases in the guide strand are modified, and all of the uracil bases in the guide strand are replaced by 5-FU. Additional compositions can include (i) an unmodified passenger strand together with a guide strand having all adenine bases replaced by 6-MP, and (ii) an unmodified guide strand and a passenger strand having all adenine bases replaced by 6-MP.

In particular compositions, the miRNA mimic can be miR-15a, miR-16, miR-129, miR-194, miR-192, miR-139, miR-140, or miR-145. Each of these miRNA mimics can include a guide strand and a passenger strand modified with 6-MP, 5-FU, or both as set forth, supra.

In a particular composition, the miRNA mimic is a miR-15a mimic that includes a guide strand consisting of the sequence UAGCAGCACAUAAUGGUUUGUG (SEQ ID NO: 1) and a passenger strand consisting of the sequence CAGGCCAUAUUGUGCUGCCUCA (SEQ ID NO: 2). In a specific composition, all six adenine bases in the guide strand and all four adenine bases in the passenger strand are replaced with 6-MP. In another specific composition, all six adenine bases in the guide strand and all four adenine bases in the passenger strand are replaced with 6-MP and all seven uracil bases in the guide strand are replaced with 5-FU.

Any of the above-described miRNA mimic compositions can be used in the method for killing a cancer cell set forth above in the SUMMARY section. In this method, the miRNA mimic is miR-15a, miR-16, miR-129, miR-194, miR-192, miR-139, miR-140, or miR-145 in which the guide strand contains both 6-MP and 5-FU and the passenger strand contains 6-MP.

The miRNA mimic compositions can also be used in the method for treating cancer disclosed above. The method features administering the miRNA mimics described above to a subject suffering from cancer. The cancers that can be treated include, but are not limited to, leukemia, lymphoma, or multiple myeloma. For example, acute myeloid leukemia (“AML”) or acute lymphocytic leukemia (“ALL”) can be treated with the disclosed method.

Administration routes of the miRNA mimic compositions include, but are not limited to oral administration, parenteral administration (e.g., subcutaneous, intramuscular, intraperitoneal, or intravenous injection), and topical administration. Additional routes that can be used are set forth in Ju.

In the cancer treatment method, the miRNA mimics can be miR-15a, miR-16, miR-129, miR-194, miR-192, miR-139, miR-140, and miR-145 in which the guide strand contains both 6-MP and 5-FU or solely 5-FU and the passenger strand contains 6-MP.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLES

### Example 1: Preparation of miRNA Mimic of miR-15a

The modified miR-15a mimic was synthesized with standard phosphoramidite chemistry as two separate RNA oligonucleotides, i.e., guide strand and passenger strand. The uracil bases were replaced with 5-FU by including in the reaction a 5-fluorouracil nucleoside phosphoramidite as a precursor, along with the phosphoramidite derivatives of nucleosides containing natural bases, e.g., A, G, and C). Similarly, a 6-MP phosphoramidite was included during synthesis with phosphoramidites containing natural C, U, and G to produce 6-MP substituted miRNA molecules.

Different combinations of unmodified and modified guide and passenger strands were produced as shown in FIGS. 1A-1F. An miR-15a mimic was produced in which the passenger strand was not modified and the guide strand was modified by replacing the six adenine bases in the guide strand with 6-MP. See FIG. 1A. Additional modified miR-15a mimics were made including a 6-MP modified passenger strand paired with an unmodified guide strand (see FIG. 1B), 6-MP modified passenger strand paired with 6-MP modified guide strand (see FIG. 1C), 6-MP modified passenger strand paired with 5-FU modified guide strand (see FIG. 1D), 6-MP modified passenger strand paired with 5-FU and 6-MP modified guide strand (see FIG. 1E), and unmodified passenger strand paired with 5-FU and 6-MP modified guide strand (see FIG. 1F).

Purified passenger and guide strands were annealed before use in transfection assays. Briefly, the passenger and guide strands were each dissolved in 100 mM acetic acid pH 3.8 to a concentration of 100 μM and mixed at a 1:1 volume ratio. The mixture was heated at 60° C. for 45 min. and then cooled at room temperature for 30 min. The annealed RNA was desalted by ethanol precipitation with 3M sodium acetate.

### Example 2: Cell Viability of Cancer Cells Exposed to Modified miRNA Mimics

### Cell Lines and Culture

MV-4-11 (American Type Culture Collection; “ATCC”) AML cells are maintained in Iscove's Modified Dulbecco's Medium (IMDM) (Thermo Fisher Scientific) supplemented with 10% fetal bovine serum (“FBS”; VWR). REH ALL cells (ATCC) are maintained in RPMI-1640 Medium (Thermo Fisher Scientific) supplemented with 10% FBS.

### Transfection

For transfection with a vehicle, twenty-four hours before transfection, cells are plated in 6 well plates at 1×10⁵ cells per well. Cells are transfected with negative control miRNA (Thermo Fisher Scientific), unmodified miR-15a, 5-FU miR-15a, or various versions of the 6-MP modified miR-15a mimic, using Lipofectamine 2000 (Thermo Fisher Scientific) as directed by the manufacturer. MV-4-11 and REH cells are transfected with an appropriate amount of miRNA as determined by pilot experiments. Twenty-four hours post-transfection, cells are replated at 2000 cells per well in 96 well plates.

For vehicle free transfection, cells are plated in 96 well plates at 2000 cells per well. Twenty-four hours later, miRNA is diluted in cell media and added to the 96 well plates.

### Cell Viability

Cell numbers are measured on days 1, 3, and 6 post transfection for cells transfected with Lipofectamine 2000 and on day 6 post transfection for vehicle free transfection, using WST-1 dye (Roche). Briefly, cells are incubated with 10 μl of WST-1 dye per 100 μl of media for 1 hour (REH) or 2 hours (MV-4-11) at 37° C. and absorbance is read at 450 nm and 630 nm. The optical density (“OD”) is calculated by subtracting the absorbance at 630 nm from that at 450 nm. OD correlates with cell viability.

### Results

miR-15a mimics modified only with 6-MP (see FIGS. 1A-1C) are more effective at inhibiting cell viability, as compared to unmodified miR-15a. miR-15a mimics that combine the 6-MP and 5-FU modifications (see FIGS. 1D-1F) are more effective than miR-15a modified with 5-FU-alone. Further, the 6-MP modified miR-15a are effective at inhibiting cell viability at concentrations lower than 50 nM.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. A composition comprising a micro-RNA mimic that contains one or more adenine bases in which at least one of the one or more adenine bases is replaced by 6-mercaptopurine.

2. The composition of claim 1, wherein the micro-RNA mimic includes a guide strand and a passenger strand.

3. The composition of claim 2, wherein the 6-mercaptopurine is present in the passenger strand.

4. The composition of claim 2, wherein the 6-mercaptopurine is present in the guide strand.

5. The composition of claim 3, wherein the 6-mercaptopurine is present in the guide strand.

6. The composition of claim 5, wherein all of the one or more adenine bases in the passenger strand are replaced with 6-mercaptopurine.

7. The composition of claim 6, wherein all of the one or more adenine bases in the guide strand are replaced with 6-mercaptopurine.

8. The composition of claim 2, wherein the guide strand contains one or more uracil bases in which at least one of the one or more uracil bases is replaced by 5-halouracil.

9. The composition of claim 8, wherein the 5-halouracil is 5-fluorouracil.

10. The composition of claim 8, wherein all of the one or more uracil bases are replaced with 5-fluorouracil.

11. The composition of claim 10, wherein the guide strand or the passenger strand contains one or more adenine bases in which all of the one or more adenine bases are replaced by 6-mercaptopurine.

12. The composition of claim 11, wherein the micro-RNA mimic is selected from the group consisting of miR-15a, miR-16, miR-129, miR-194, miR-192, miR-139, miR-140, and miR-145.

13. The composition of claim 12, wherein the guide strand has the sequence of UAGCAGCACAUAAUGGUUUGUG (SEQ ID NO: 1) and the passenger strand has the sequence of CAGGCCAUAUUGUGCUGCCUCA (SEQ ID NO: 2).

14. A method for killing a cancer cell, comprising contacting the cancer cell with the composition of claim 12.

15. A method for treating cancer, the method comprising identifying a subject suffering from cancer and administering to the subject an effective amount of a composition containing a micro-RNA mimic having a guide strand and a passenger strand that each contains one or more adenine bases in which at least one adenine base in the guide strand or at least one adenine base in the passenger strand is replaced by 6-mercaptopurine.

16. The method of claim 15, wherein the cancer is leukemia, lymphoma, or multiple myeloma.

17. The method of claim 16, wherein all of the one or more adenine bases in the guide strand or in the passenger strand are replaced with 6-mercaptopurine.

18. The method of claim 17, wherein the guide strand contains one or more uracil bases in which at least one uracil base is replaced with 5-fluorouracil.

19. The method of claim 18, wherein all of the one or more uracil bases are replaced with 5-fluorouracil.

20. The method of claim 19, wherein the micro-RNA mimic is selected from the group consisting of miR-15a, miR-16, miR-129, miR-194, miR-192, miR-139, miR-140, and miR-145.
